(19) United States
(12) Patent Application Publication
(10) Pub. No.: US 2014/0249087 A1
(43) Pub. Date: Sep. 4, 2014

(54) USE OF HEMOPEXIN TO SEQUESTER HEMOGLOBIN

(71) Applicant: The General Hospital Corporation, Boston, MA (US)

(72) Inventor: H. Shaw Warren, Cambridge, MA (US)

(21) Appl. No.: 14/347,856

(22) PCT Filed: Sep. 27, 2012
(86) PCT No.: PCT/US2012/057519

§ 371 Date: Mar. 27, 2014

Related U.S. Application Data
(60) Provisional application No. 61/651,349, filed on May 24, 2012.
(60) Provisional application No. 61/540,309, filed on Sep. 28, 2011.

(51) Int. Cl. A61K 38/17

(52) U.S. Cl. CPC A61K 38/1709
USPC 514/15.3; 435/2

(57) ABSTRACT

The invention relates to use of hemopexin (Hx) to sequester extravascular hemoglobin and thereby reduce or prevent inflammation of non-infectious etiology in a subject (e.g., a human).

### STATEMENT AS TO FEDERALLY FUNDED RESEARCH

This invention was made with government support under RO1AI59010 awarded by the National Institutes of Health. The government has certain rights in the invention.

### BACKGROUND OF THE INVENTION

The invention relates to use of hemopexin (Hx) to reduce inflammation or reduce the risk of developing inflammation.

The release of blood into non-vascular tissue can occur as a result of trauma, as well as in numerous disorders. Such release of blood can lead to inflammation which, in turn, can result in destruction of healthy tissue and thereby exacerbate the trauma or negative effects caused by the disorder.

Traumatic brain injury (TBI), for example, affects over 1.7 million people each year in the United States alone. The estimated economic cost of TBI in the U.S. in 2010 was $76.5 billion. Trauma, such as a TBI, is also a common cause of hemorrhagic stroke, which affects approximately 120,000 people in the

U.S. alone. Hypertension, aneurysm, cavernous malformations, amyloid angiopathy, arteriovenous malformation (AVM), and brain tumors can also result in hemorrhagic stroke.

Preterm births (births before completion of 37 weeks of gestation), which account for approximately 8 to 10% of all pregnancies in the U.S., often result in premature infants who are at greater risk for both short- and long-term complications. Premature infants often have a high incidence of inflammation (e.g., inflammation of the lungs), infection (e.g., sepsis), and neurological problems (e.g., retinopathy of prematurity (ROP)). Worldwide, premature births result in approximately 500,000 deaths per year. Many of these complications result from release of blood into non-vascular tissue.

Ocular trauma and numerous eye diseases also result in the release of blood and blood breakdown products into non-vascular tissue. For example, age-related macular degeneration (AMD), myopia, and ocular trauma are associated with choroidal neovascularization, which can result in blindness due to tissue destruction caused by hemorrhaging. AMD, in particular, is the leading cause of vision loss in Americans 60 years of age and older.

Thus, there is a need to identify methods to better treat and control inflammation resulting from the release of blood into non-vascular tissue to reduce destruction of healthy tissue.

### SUMMARY OF THE INVENTION

The present invention provides methods for the use of hemopexin (Hx) in the treatment and prevention of inflammation. In particular, the present invention relates to methods of sequestering extravascular or extracellular hemoglobin (Hb) in a subject by administration of Hx and thereby reducing inflammation or reducing the risk of developing inflammation. Inflammation that can be treated or prevented by administration of Hx need not be caused by an infectious agent, but rather may be the result of non-infectious etiology (e.g., trauma or preterm birth).

Accordingly, in the first aspect, the invention features a method of sequestering extravascular Hb. This method involves administering human Hx, or an amino acid sequence that is at least 95% identical to human Hx, to a subject (e.g., a human) in an amount sufficient to sequester at least 20% of the extravascular Hb, where the sequestering reduces inflammation in the subject or reduces the risk of the subject developing inflammation. In a desirable embodiment of the first aspect of the invention, the subject is a premature infant.

In another desirable embodiment, human Hx is administered to the subject. In further desirable embodiments of the first aspect of the invention, the administered human Hx sequesters at least 30%, 40%, 50%, 60%, 70%, 80%, or 90% of the extravascular Hb.

In additional desirable embodiments of the first aspect of the invention, the subject has suffered trauma (e.g., acute lung injury, ocular trauma, head trauma, e.g., traumatic brain injury (TBI)), has suffered a stroke (e.g., hemorrhagic stroke), has extravascular Hb present in an eye (e.g., an eye disease associated with choroidal neovascularization, such as, diabetic retinopathy, age-related macular degeneration (AMD), myopia), has asthma, has necrotic tissue, or is suffering from acute chest syndrome, sickle cell anemia, hemophagocytic lymphohistiocytosis (HLH), paroxysmal noctural hemaglobinuria (PNH), hemolytic uremic syndrome (HUS) or thrombotic thrombocytopenic purpura (TTP).

In further desirable embodiments, Hx is administered directly to the site containing extravascular Hb (e.g., the eye or necrotic tissue). Administration, in a desirable embodiment, is by direct injection.

In other desirable embodiments of the first aspect of the invention, the mode of administration is parenteral administration or topical administration.

In the second aspect, the invention features a method of sequestering extracellular Hb in a subject having sickle cell anemia. This method involves administering human Hx, or an amino acid sequence that is at least 95% identical to human Hx, to the subject (e.g., a human) in an amount sufficient to sequester at least 20% of the extracellular Hb, where the sequestering reduces inflammation in the subject or reduces the risk of the subject developing inflammation.

In desirable embodiments, a disease of non-infectious etiology that can be treated or prevented by administration of Hx is not an autoimmune disease.

In the third aspect, the invention features a method of removing extracellular or extravascular hemoglobin or heme from blood or a tissue. This method involves contacting the blood or tissue with a substrate containing human hemopexin, thereby removing the extracellular or extravascular hemoglobin or heme from the blood or tissue.

In a desirable embodiment of the third aspect of the invention, the extracellular hemoglobin is removed from the blood prior to or during a blood transfusion, and in another desirable embodiment of the third aspect of the invention, the extracellular or extravascular hemoglobin is removed from the blood or tissue to treat an inflammatory response in a subject.

### Definitions

By “sequestering” hemoglobin (Hb) is meant forming a complex of extravascular or extracellular Hb and Hx or an amino acid sequence that is at least 95% identical to human Hx. Sequestration of Hb may be measured directly, by detecting complexes of Hb and Hx using standard techniques for detecting protein interactions, or may be measured indirectly by observing a reduction or elimination of an inflammatory response due to a disorder that results in release of Hb into non-vascular tissue. Examples, of such disorders include necrosis-related inflammation, trauma (e.g., acute lung injury, ocular trauma, head trauma, e.g., traumatic brain injury (TBI)), stroke (e.g., hemorrhagic stroke), asthma, sickle cell anemia, hemophagocytic lymphohistiocytosis (HLH), paroxysmal noctural hemaglobinuria (PNH), hemolytic uremic syndrome (HUS) or thrombotic thrombocytopenic purpura (TTP), eye diseases associated with choroidal neovascularization (e.g., diabetic retinopathy, age-related macular degeneration (AMD), myopia), and premature infants with inflammation (e.g., inflammation of the lungs, retinopathy of prematurity (ROP)).

By an “amount sufficient” is meant an amount of human Hx, or an amino acid sequence that is at least 95% identical to human Hx, that is capable of effecting beneficial or desired results, such as a reduction or elimination of an inflammatory response. For example, in the context of administering a composition that sequesters extravascular or extracellular Hb, the amount sufficient is an amount sufficient to achieve sequestration of at least 20% of the extravascular or extracellular Hb, as compared to the response obtained in the absence of the administration of the composition. Desirably, 30%, 40%, 50%, 60%, 70%, 80%, 90%, or even 95% of extravascular Hb is sequestered by human Hx or an amino acid sequence that is at least 95% identical to human Hx. Desirably, sequestration of Hb by Hx leads to a 30%, 40%, 50%, 70%, 80%, 90%, or even 95% reduction of an inflammatory response. A reduction of an inflammatory response may be measured by a reduction of the level, in a subject, of one or more pro-inflammatory cytokines such as tumor necrosis factor (TNF), IL-6, or IL-1 relative to a control (e.g., a sample from the subject prior to administration of Hx). Desirably, the level of the pro-inflammatory cytokine is reduced by 30%, 40%, 50%, 70%, 80%, 90%, or even 95% relative to the control.

By “extravascular” Hb or heme is meant Hb or heme that is located outside of a blood vessel or lymph vessel.

By “extracellular” Hb or heme is meant Hb or heme that is located outside of a red blood cell.

By “human hemopexin” or “human Hx” is meant a polypeptide with the amino acid sequence of GenBank Accession No. AAA58678 (Homo sapiens) or NP_{—}000604 (Homo sapiens).

By “inflammatory response” is meant the activation of the immune system in a subject. Desirably the subject is a mammal such as a human. An inflammatory response preferably involves the induction of cytokines and may result from an autoimmune disease such as rheumatoid arthritis or inflammatory bowel disease, or from contact of the mammal with a virus, a gram-negative bacterium, a gram-positive bacterium, or component thereof, such as lipopolysaccharide (LPS).

By “% identity” or “% identical” is meant a polypeptide sequence that has a specified percentage of amino acid residues that are the same at the corresponding location within a reference sequence when the two sequences are optimally aligned. For example, an amino acid sequence that is at least “95% identical” to a reference sequence has at least 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, or 20 contiguous amino acids, more preferably at least 25, 50, 75, 90, 100, 150, 200, 250, 300, or 350 contiguous amino acids, and most preferably the full-length amino acid sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

A “subject” is a vertebrate, such as a mammal, e.g., a human. Mammals include, but are not limited to, farm animals (such as cows), sport animals, pets (such as cats, dogs, and horses), mice, rats, and primates. Desirably, the subject is a human such as an adult human or a premature infant.

The term “removing” as used herein in connection with the phrase “removing extracellular or extravascular Hb (or heme) from blood or a tissue” means that at least 20%, 30%, 40%, 50%, 60%, 70%, 80%, 90%, or even 95% of extracellular or extravascular Hb or heme is bound by human Hx on a substrate and thereby removed from the blood or tissue.

Throughout this specification and claims, the word “comprise,” or variations such as “comprises” or “comprising,” will be understood to imply the inclusion of a stated integer or group of integers but not the exclusion of any other integer or group of integers.

### BRIEF DESCRIPTION OF THE DRAWINGS

FIG. 1A is a graph showing that hemoglobin (Hb) synergizes with high-mobility group protein B1 (HMGB1) to induce tumor necrosis factor (TNF) production by macrophages. The results represent mean±standard error and are representative of more than three independent experiments. *=P<0.05, **=P<0.01, compared between cells treated with and without Hb.

FIG. 1B is a graph showing that Hb synergizes with HMGB1 to induce IL-6 production by macrophages. The results represent mean±standard error and are representative of more than three independent experiments. *=P<0.05, **=P<0.01, compared between cells treated with and without Hb.

FIG. 1C is a graph showing that Hb synergizes with HMGB1 to induce TNF production by macrophages. The results represent mean±standard error and are representative of more than three independent experiments. *=P<0.05, **=P<0.01, compared between cells treated with and without Hb.

FIG. 1D is a graph showing that Hb synergizes with HMGB1 to induce IL-6 production by macrophages. The results represent mean ±standard error and are representative of more than three independent experiments. *=P<0.05, **=P<0.01, compared between cells treated with and without Hb.

FIG. 2A is a graph showing that the synergy of Hb with HMGB1 is not completely dependent on Toll-like receptor 2 (TLR2). The results represent mean±standard error and are representative of four independent experiments. **=P<0.01, ***=P<0.001 compared between cells treated with and without Hb.

FIG. 2B is a graph showing that the synergy of Hb with HMGB1 is not completely dependent on Toll-like receptor 4 (TLR4). The results represent mean±standard error and are representative of four independent experiments. *=P<0.05, compared between cells treated with and without Hb.

FIGS. 3A and 3B are graphs showing the effect of hemopexin (Hx) on hemoglobin synergy with HMGB1 on bone marrow-derived macrophages (BMDMs). The results represent mean±standard error and are representative of four independent experiments. **=P<0.01, compared between cells treated with and without Hx.

FIG. 4A is a graph showing the synergistic inflammation induced by Hb, HMGB1, and LPS. The results represent mean±standard error and are representative of three independent experiments. **=P<0.01, compared between cells treated with three stimuli and with one or two stimuli.

FIG. 4B is a graph showing the synergistic inflammation induced by Hb, HMGB1, and P3C. The results represent mean±standard error and are representative of three independent experiments. **=P<0.01, compared between cells treated with three stimuli and with one or two stimuli.

FIG. 5A is a graph showing the effect of Hx on the synergy induced by Hb, HMGB1, and LPS (lipopolysaccharide). The results represent mean±standard error and are representative of three independent experiments. **=P<0.01, compared between cells treated with or without Hx.

FIG. 5B is a graph showing the effect of Hx on the synergy induced by Hb, HMGB1, and P3C (Pam3Cys). The results represent mean±standard error and are representative of three independent experiments. **=P<0.01, compared between cells treated with or without Hx.

### DETAILED DESCRIPTION

The present invention is based on the inventor's discovery that hemopexin (Hx) is involved in the regulation of an inflammatory response, even in the absence of an infection. Induction of inflammation is an essential component of innate immunity and can be triggered by infectious (e.g., microbial) or non-infectious etiologies (e.g., trauma, acute lung injury, stroke). Generation of an inflammatory response depends on the production of pro-inflammatory cytokines (e.g., TNF or IL-6) by macrophages. In some cases, the inflammatory response can be overactive and result in tissue damage and sepsis. It is therefore critical that the inflammatory response is regulated. The discovery disclosed herein enables the use of Hx in the treatment or prevention of inflammation resulting from non-infectious etiologies. In particular, administration of Hx to a subject results in sequestration of extravascular or extracellular Hb and, thereby, reduces or prevents an inflammatory response.

### Inflammation of Non-Infectious Etiology

Inflammation can be associated with non-infectious etiologies that result in the release of blood or blood breakdown products into non-vascular tissue. Examples of inflammation of non-infectious etiology include necrosis-related inflammation, trauma (e.g., acute lung injury, ocular trauma, head trauma, e.g., traumatic brain injury (TBI)), stroke (e.g., hemorrhagic stroke), asthma, sickle cell anemia, hemophagocytic lymphohistiocytosis (HLH), paroxysmal noctural hemaglobinuria (PNH), hemolytic uremic syndrome (HUS), or thrombotic thrombocytopenic purpura (TTP), eye diseases associated with bleeding in the eye (e.g., eye disease associated with choroidal neovascularization, such as, diabetic retinopathy, age-related macular degeneration (AMD), myopia), and premature infants with inflammation (e.g., inflammation of the lungs, retinopathy of prematurity (ROP)).

### Methods of Treatment of Inflammation Using Hemopexin

We have shown that Hx, the primary heme scavenger in plasma, is able to significantly block the synergy of hemoglobin (Hb) with LPS. Hb is a globular heme-binding protein that is present in the red blood cells of nearly all vertebrates. In humans and most vertebrates, hemoglobin is a heterotetrameric protein. In adult humans, hemoglobin is predominantly formed by two a subunits and two β subunits non-covalently bound to one another, each subunit including a non-protein heme group. In human infants, hemoglobin is predominantly formed by two a subunits and two y subunits non-covalently bound to one another, each subunit including a non-protein heme group.

In addition, Hx downregulates pro-inflammatory cytokines (e.g., TNF or IL-6) from macrophages (Liang et al., J. Leukoc. Biol., 86: 229-235, 2009) and functions as an anti-inflammatory component of serum HDL in atherosclerosis (Watanabe et al., J. Biol. Chem., 284: 18292-18301, 2009). We have now further characterized the functions of Hx in an inflammatory response. We find that Hb and the endogenous molecule, high-mobility group protein B1 (HMGB1), synergize to induce pro-inflammatory cytokine production by macrophages, and that this synergy is blocked by Hx.

We have also found that Hx can bind Hb and not just heme as previously reported. Thus, administration of Hx can be used to bind extravascular or extracellular Hb and reduce the pro-inflammatory effect resulting from the synergy of Hb with HMGB1. The invention therefore relates to the treatment of a subject having an inflammatory response of non-infectious etiology by administration of Hx to sequester extravascular or extracellular Hb. Inflammation in the subject can be caused by, or associated with, a non-infectious etiology such as trauma, necrosis, preterm birth, capillary breakdown, or any other process that results in the release of blood into non-vascular tissue in the subject.

### Methods of Using Hx to Remove Extracellular or Extravascular Hb or Heme

Having shown that Hx can bind Hb, Hx can also be used to bind extracellular or extravascular Hb ex vivo. Blood or tissue containing or likely to contain extracellular or extravascular Hb can be contacted with Hx, for example, Hx bound to a substrate, to bind the extracellular or extravascular Hb and, thereby remove it from the blood or tissue. Similarly, Hx, in such methods, could be used to remove extracellular or extravascular heme, heme containing molecules, or a heme containing complex from the blood or tissue.

Methods of removing extracellular or extravascular Hb or heme from blood or a tissue can be used in conjunction with blood transfusions, blood purification, or in the treatment of any disorder that results in the release of Hb from red blood cells or in the release of blood into non-vascular tissue in a subject.

Moreover, removing extracellular or extravascular Hb or heme from blood or a tissue may be used in methods of treating an inflammatory response in a subject, such as inflammation resulting from an autoimmune disease such as rheumatoid arthritis or inflammatory bowel disease, or from contact of the subject with a virus, a gram-negative bacterium, a gram-positive bacterium, or component thereof, such as lipopolysaccharide (LPS). Removing extracellular or extravascular Hb or heme from blood or a tissue also may be used in treating an inflammatory response in a subject that results in sepsis.

Extracellular or extravascular Hb or heme may be removed from blood or a tissue by contacting a substrate (e.g., a membrane or filter) containing bound Hx with the blood or tissue. Substrates for binding proteins such as Hx are known in the art and include, for example, membranes such as nitrocellulose and PVDF (polyvinylidene fluoride). Hx may also be bound to a protein chip or microarray using standard methods. Protein chip and microarray technology is well known in the art and is described in Protein Microarray Technology, Kambhampati (ed.) 2004. The substrate containing bound Hx may be included in a medical device used in connection with blood transfusions or blood purification.

### Administration

The methods described herein include administration of Hx by a route selected from, e.g., ocular, topical, parenteral, dermal, transdermal, inhalation, buccal, sublingual, perilingual, nasal, rectal, and oral administration. Parenteral administration includes intravenous, intraperitoneal, subcutaneous, and intramuscular administration. Thus, Hx may be injected systemically, for example, by the intravenous injection of Hx into the subject's bloodstream or alternatively, the compound can be directly injected at the site of an inflammation. Intraocular, parenteral, or intranasal administration may be provided by using, e.g., aqueous suspensions, isotonic saline solutions, sterile and injectable solutions containing pharmacologically compatible dispersants and/or solubilizers, for example, propylene glycol or polyethylene glycol, lyophilized powder formulations, and gel formulations. The preferred method of administration can vary depending on various factors (e.g., the components of the composition being administered and the severity of the condition being treated). Hx can be formulated for topical administration in a composition in the form of oil, cream, ointment and the like. Suitable carriers for the composition include vegetable or mineral oils, white petrolatum (white soft paraffin), branched chain fats or oils, animal fats, and high molecular weight alcohols (greater than C12). The preferred carriers are those in which Hx is soluble. Emulsifiers, stabilizers, humectants and antioxidants may also be included, as well as agents imparting color or fragrance, if desired. Formulations suitable for oral or nasal administration may consist of liquid solutions, such as an effective amount of the composition dissolved in a diluent (e.g., water, saline, or PEG-400), capsules, sachets, tablets, or gels, each containing a predetermined amount of Hx. Hx may also be administered in an aerosol formulation for inhalation, e.g., to the bronchial passageways. Aerosol formulations may be mixed with pressurized, pharmaceutically acceptable propellants (e.g., dichlorodifluoromethane, propane, or nitrogen). In particular, administration by inhalation can be accomplished by using, e.g., an aerosol containing sorbitan trioleate or oleic acid, for example, together with trichlorofluoromethane, dichlorofluoromethane, dichlorotetrafluoroethane, or any other biologically compatible propellant gas.

Pharmaceutical compositions of Hx described herein may be formulated to release the composition immediately upon administration (e.g., targeted delivery) or at any predetermined time period after administration using controlled or extended release formulations. Administration of the pharmaceutical Hx composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window at the site of release; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

Hemopexin may be administered to provide prophylaxis to or reduce inflammation in a subject. Hemopexin may be administered, e.g., 1, 2, 3, 4, 5, 6, 7, 8, 9, 10, 15, 20, 30, 35, 40, 45, 50, 55, or 60 minutes, 2, 4, 6, 10, 15, or 24 hours, 2, 3, 5, or 7 days, or 2, 4, 6 or 8 weeks prior to an inflammation-stimulating event (e.g., prior to exercise for an exercise-induced asthmatic, prior to allergen exposure for an allergy-induced asthmatic), or may be administered to the subject 1, 2, 3, 4, 5, 6, 7, 8, 9, 10, 12, 20, 24, 48, 72 hours, or longer after an inflammatory response has occurred (e.g., after trauma or stroke).

When treating inflammation, Hx may be administered to the subject either before the occurrence of symptoms or a definitive diagnosis or after diagnosis or symptoms of inflammation become evident. For example, Hx may be administered, e.g., immediately after diagnosis or the clinical recognition of symptoms or 2, 4, 6, 10, 15, or 24 hours, 2, 3, 5, or 7 days, or 2, 4, 6 or 8 weeks after diagnosis or detection of symptoms.

Hemopexin may be sterilized by conventional sterilization techniques, or may be sterile filtered. The resulting aqueous solutions may be packaged for use as is or lyophilized. The lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of Hx, such as in a sealed package of tablets or capsules, or in a suitable dry powder inhaler (DPI) capable of administering one or more doses.

### Dosages

The dose of Hx or the number of treatments using Hx may be increased or decreased based on the severity of, occurrence of, or progression of, the inflammation in the subject.

The pharmaceutical compositions of Hx can be administered in a therapeutically effective amount that provides a protective effect against inflammation. For example, a dosage regimen of Hx can be administered from 1 mg to 2000 mg/day, preferably 1 to 1000 mg/day, more preferably 50 to 600 mg/day, in one or more divided doses.

The dosage of Hx administered depends on the subject to be treated (e.g., the age, body weight, capacity of the immune system, and general health of the subject being treated), the form of administration (e.g., as a solid or liquid), the manner of administration (e.g., by injection, inhalation, dry powder propellant), and the cells targeted (e.g., epithelial cells, such as blood vessel epithelial cells, nasal epithelial cells, or pulmonary epithelial cells). The composition is preferably administered in an amount that provides a sufficient level of human Hx, or an amino acid sequence that is at least 95% identical to human Hx, that reduces or prevents inflammation without undue adverse physiological effects in the subject caused by the treatment.

In addition, single or multiple administrations of Hx may be given to a subject (e.g., one administration or administration two or more times). Reduced levels of inflammation provided by the pharmaceutical compositions of Hx described herein can be monitored by, e.g., measuring amounts of pro-inflammatory cytokines, e.g., TNF or IL-6, by standard techniques, e.g., ELISA. The dosages may then be adjusted or repeated as necessary.

A single dose of Hx may achieve protection prior to inflammation. In addition, a single dose of Hx administered post-inflammatory response can function as a treatment according to the present invention.

A single dose of Hx can also be used to achieve therapy in subjects being treated for a inflammation. Multiple doses (e.g., 2, 3, 4, 5, or more doses) can also be administered, in necessary, to these subjects.

### Carriers, Excipients, Diluents

Therapeutic formulations of Hx are prepared using standard methods known in the art by mixing the active ingredient having the desired degree of purity with optional physiologically acceptable carriers, excipients, or stabilizers (Remington's Pharmaceutical Sciences (21^{th} edition), ed. A. Gennaro, 2005, Lippincott, Williams & Wilkins, Philadelphia, Pa.). Acceptable carriers, include saline or buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone, amino acids such as glycine, glutamine, asparagines, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG (polyethyleneglycol).

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

### EXAMPLES

The following examples are meant to illustrate the invention and should not be construed as limiting.

### Example 1

### Materials and Methods

The experiments described herein may be carried out using the following materials and methods.

Materials

The following TLR agonists were purchased: smooth LPS from E. coli O55:B5 (List Biologicals), Pam3Cys (EMC Microcollections). All TLR agonists were dissolved in pyrogen-free H₂O and saved as aliquots at −80° C. C57BL/6, TLR2 knockout, C3H/HeN, and C3H/HeJ mice were obtained from Charles River Labs. Recombinant HMGB1 was expressed in E. coli and purified to homogeneity as previously described (Wang et al., Science, 285: 248-251, 1999; Li et al., J. Immunol. Meth., 289: 211-223, 2004). Briefly, HMGB-1 was cloned by DNA amplification from Rat Brain Quick-Clone cDNA (Clontech, Palo Alto, Calif.). The PCR product was subcloned into the pCAL-n vector with a calmodulin binding protein (CBP) tag (Stratagene, La Jolla, Calif.).

Limulus Amoebocyte Lysate (LAL) Assay

The LAL assay was performed as previously described (Novitsky et al., J. Clin. Microbiol., 21: 211-216, 1985) to verify the endotoxin level to be lower than 0.01 EU/mg in HMGB1, purified Hb and Hx before used in cell cultures.

Purification of Mouse and Human Hemoglobin

Hemoglobin was purified as previously described with modifications (Andrade et al., Int. J. Biol. Macromol., 34: 233-240, 2004), utilizing pyrogen-free conditions. Briefly, mouse blood was collected from C57BL/6 mice by cardiac puncture. Human blood was collected aseptically from healthy human volunteers. The blood was washed with an equal weight of isotonic saline solution (0.9% NaCl, w/v) three times by centrifugation at 1,000×g to remove serum proteins. Equal volumes of saline were added to the pellet containing red blood cells and this solution was sonicated 5×10 seconds at amplitude 40% with 1-minute laps between pulses in Branson 450 sonicator from Branson Ultrasonics Corporation (Danbury, Conn.). The Hb solution was diluted with an equal volume of saline and subjected to a second centrifugation at 2,000×g for 1 hour. The resulting Hb solution, removed from the center layer, was filtered through 0.22-μm Millipore membranes and saved at −20° C. in the dark. The concentration of purified mouse Hb was measured by Micro-BCA. The purity of the Hb was confirmed to be >99% by non-denaturing PAGE and high pressure liquid chromatography.

Purification of Hemopexin from Mouse or Human Serum

Mouse serum Hx (mHx) or human serum Hx (hHx) was purified using heme affinity chromatography essentially as we have described (Liang et al., J. Leukoc. Biol., 86: 229-235, 2009). Briefly, after filtration through 0.22-μm Millipore membranes, the abundant albumin in the serum was precipitated and removed by cold 1.68% rivanol solution (pH 8.0). The post rivanol-precipitation sample was dialyzed against pyrogen-free PBS. Protease inhibitors (0.5 mM AEBSF, 10 μM E-64, 2 μg/ml aprotinin and 1 μM pepstatin A) were added and interacted with the dialyzed post rivanol-precipitation for 15 minutes by gentle agitation at 4° C. The mixture was applied to a 6-ml hemin-agarose column (Sigma) for 3 times, followed by extensive washing with 1,200 ml PBS containing 0.5M NaCl overnight at 4° C. to remove unbound proteins. Hx bound to the column was eluted by 0.2 M citric acid (pH 4.0) followed by immediate neutralization with 10 M NaOH. Proteins in the buffer were exchanged, concentrated in PBS at 4° C. by using Centriprep YM-30 (Millipore, Mass.), and saved in aliquots at −80° C.

Preparation of Macrophages

Bone marrow-derived macrophages (BMDMs) were prepared from mice, as described (Schilling et al., J. Immunol., 169: 5874-5880, 2002) with minor modifications (Bagchi et al., J. Immunol., 178: 1164-1171, 2007; Liang et al., J. Leukoc. Biol., 86: 229-235, 2009). BMDMs were seeded at 1.28×10⁵/well in 96 well-tissue culture plates and allowed to adhere for overnight before use in assays.

Macrophage Culture and Cytokine Assays

BMDMs were washed 3 times in serum-free medium, followed by incubation overnight with HMGB1, with or without Hb, or with different TLR agonists as desired at indicated concentrations. Purified mHx (mouse Hx) was added to the culture in some experiments as noted. Levels of TNF and IL-6 in the supernatants were quantitated by enzyme-linked immunosorbent assay (ELISA) (R&D Systems, Minneapolis, Minn.) according to the manufacturer's instructions.

Statistics

Except where indicated, representative data from at least three experiments are presented in the figures. Data are expressed as means, and error bars represent standard error. The data were analyzed by GraphPad Prism 5 (GraphPad software, La Jolla, Calif.). Values of p<0.05 were considered to be statistically significant.

### Example 2

### Hemoglobin Strongly Synergizes with HMGB1 to Induce TNF and IL-6 from Macrophages

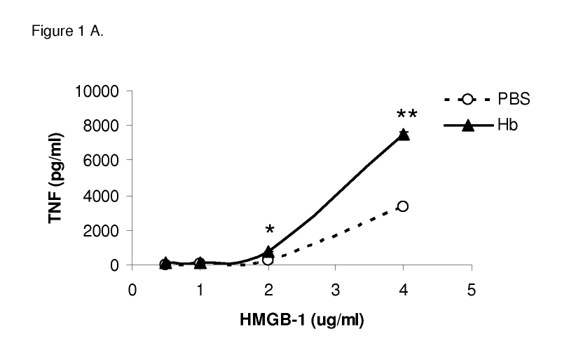

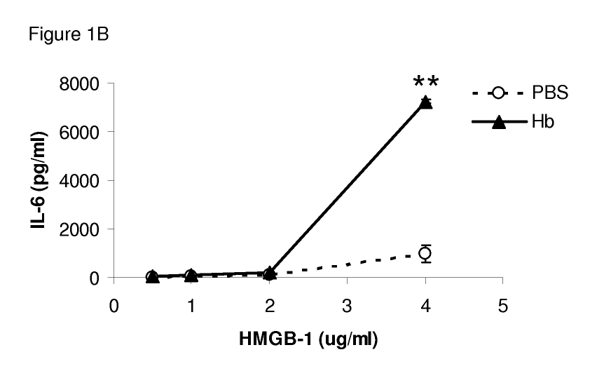

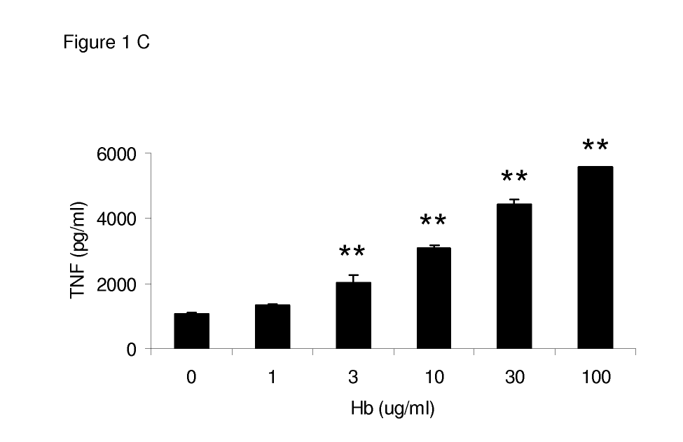

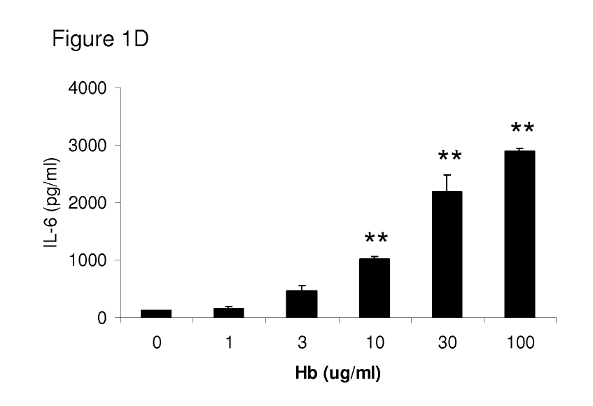

Different concentrations of HMGB1 were incubated with a predetermined optimized concentration of mouse Hb in cell culture with BMDMs. Bone marrow-derived macrophages (BMDMs) from C57BL/6 mice were cultured with HMGB1 at different concentrations with mouse Hb (30 82 g/ml). Pro-inflammatory cytokines TNF and IL-6 levels in the supernatant were measured by ELISA. HMGB1 at concentrations above 1 μg/ml induced low levels of TNF (FIG. 1). Hemoglobin alone could not induce detectable TNF at concentrations up to 1,000 μg/ml (Lin et al., J. Infect. Dis. 2010, 202: 624-632), but significantly enhanced the production of TNF from BMDMs by HMGB1 at different concentrations (FIG. 1A), and this effect was dose dependent (FIG. 1C). Similar results were found with IL-6 induced in the culture (FIGS. 1B and 1D). Human Hb (hHb) also synergized with HMGB1 to induce high levels of TNF and IL-6.

### Example 3

### Synergistic Induction of Pro-Inflammatory Cytokines by HMGB1 and Hemoglobin are Partially Dependent of TLR2 and TLR4

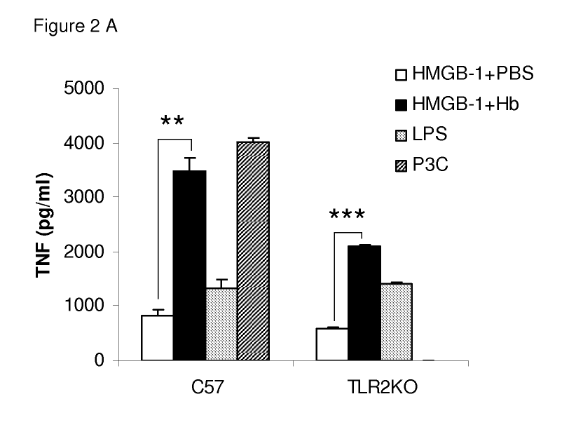

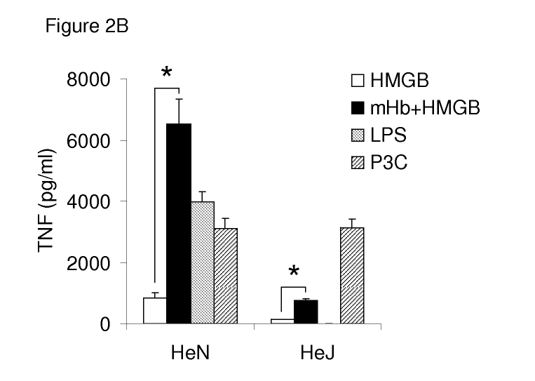

Because HMGB1 binds to TLR4 and probably TLR2 to activate macrophages, we addressed the question of whether the synergy of Hb with HMGB1 works through TLR4 or TLR2 signaling pathways. For these experiments, the synergistic induction of cytokines induced in BMDMs from TLR2 knockout (TLR2KO) mice was compared with induction of cytokines from control wild-type C57/BL6 mice (FIG. 2A), while C3H/HeJ mice, which are deficient in TLR4, was compared with induction of cytokines from control BMDMs from C3H/HeN mice stimulated with HMGB1 in the absence or presence of hemoglobin (FIG. 2B). BMDMs from C57BL/6 (control) and TLR2 knockout (TLR2KO) mice, or BMDMs from HeN (control) and HeJ mice (deficient for TLR4), were cultured with Pam3Cys (P3C), LPS, or HMGB1 (4 μg/ml) with or without Hb (30 μg/ml). Concentrations of TNF in the supernatant were determined by ELISA. TLR2 agonist P3C and TLR4 agonist LPS were used as controls to stimulate different cell types. P3C could induce TNF in C57/BL6 cells but not from TLR2KO cells (FIG. 2A), which was expected, while LPS could induce same amount of TNF on both cell types, indicating that equal number of both cell types in the culture (FIG. 2A). Similarly, in FIG. 2B, LPS could induce TNF in C3H/HeN cells but not from C3H/HeJ cells, which was expected, while P3C could induce same amount of TNF in both cell types. The synergy for HMGB1 with Hb was present despite the deficiency of TLR2 (FIG. 2A) or TLR4 (FIG. 2B), suggesting that ligation of TLR4 or TLR2 are not required for the synergy, although TLR4 signaling may be dominant.

### Example 4

### Hemopexin Blocks the Synergistic Induction of Pro-Inflammatory Cytokines from Macrophages by HMGB1 and Hemoglobin

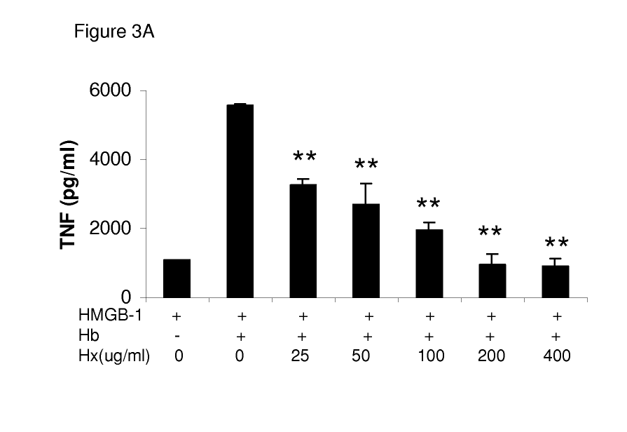

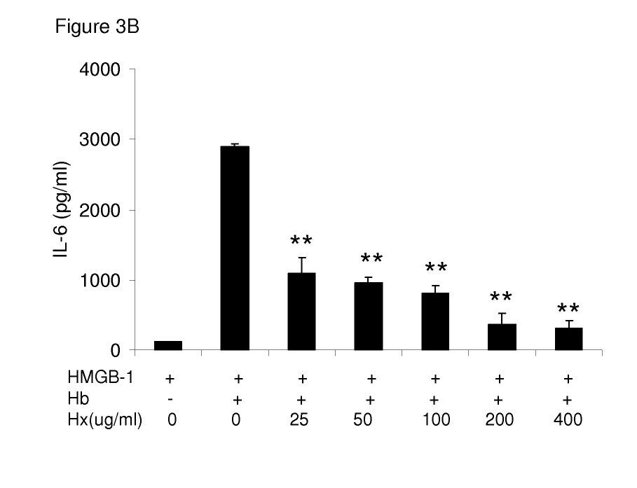

We have shown that, Hx, the major heme scavenger in the plasma is able to significantly block the synergy of hemoglobin with LPS (Lin et al., J. Infect. Dis. 202: 624-632, 2010). In addition, Hx has some immunomodulatory activities and modestly downregulates pro-inflammatory cytokines from macrophages (Liang et al., J. Leukoc. Biol., 86: 229-235, 2009). Hemopexin also functions as an anti-inflammatory component of serum HDL in atherosclerosis (Watanabe et al., J. Biol. Chem., 284: 18292-18301, 2009). It was therefore of interest to assess if Hx would also affect the synergistic induction of pro-inflammatory cytokines that were induced by Hb with HMGB1. BMDMs from C57BL/6 mice were cultured with HMGB1 (4 μg/ml) with or without mouse Hb (30 μg/ml) and different concentrations of Hx. Concentrations of TNF and IL-6 in the supernatants were determined by ELISA. We found that Hx dramatically suppressed the synergistic induction of TNF (FIG. 3A) and IL-6 (FIG. 3B) in a dose-dependent manner.

### Example 5

### TLR2 and TLR4 Agonists Synergize with HMGB1 and Hemoglobin to Induce Significant High Levels of TNF in Macrophages Suppressed by Hemopexin

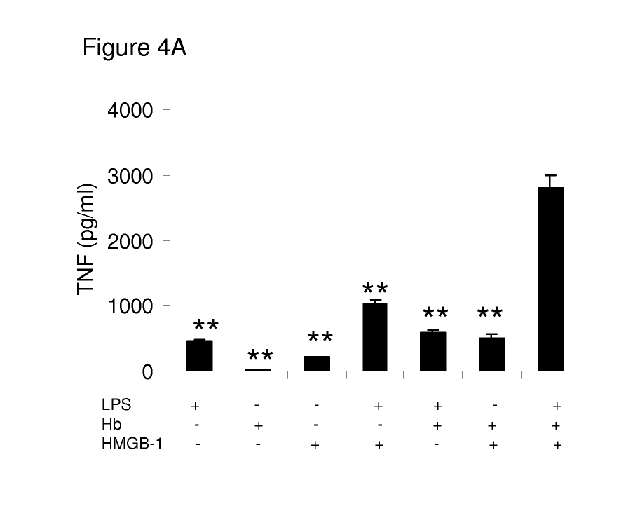

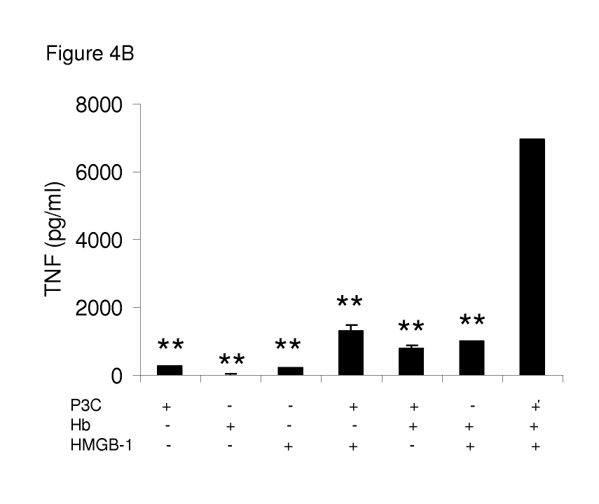

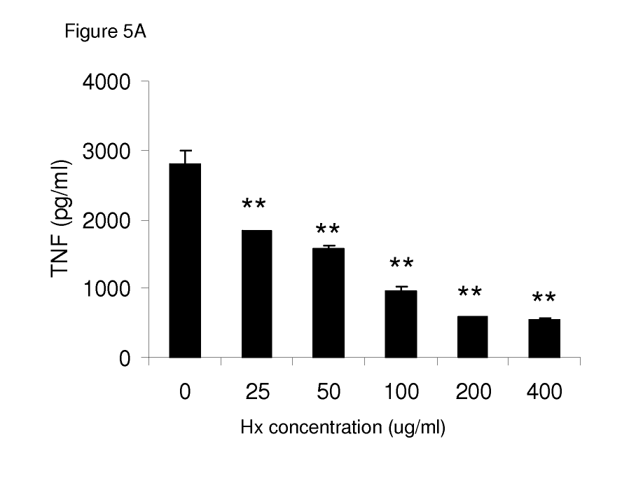

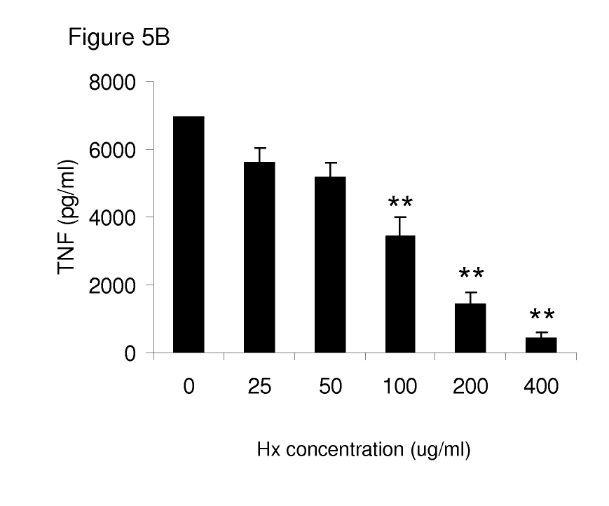

Because it is common that tissue damage, necrosis, and bleeding co-exist in some settings of infection, we tested if there is a more significant synergy with three types of stimuli including HMGB1, Hb, and microbial TLR agonists such as LPS. Low concentrations of LPS (250 pg/ml), HMGB1 (2 μg/ml), and Hb (10 μg/ml) were used to stimulate BMDMs. Dramatically higher levels of TNF were observed in the culture after overnight incubation of all three stimuli compared to the cultures with a single stimulus or a combination of two stimuli (FIG. 4A). When TLR2 agonist P3C (1 ng/ml) instead of LPS was used, the same phenomena were observed (FIG. 4B). When Hx was added to the culture, the synergy with all three types of stimuli was remarkably suppressed in the case of either combination of HMGB1, Hb, and LPS (250 pg/ml) (FIG. 5A) or HMGB1, Hb, and P3C (1 ng/ml) (FIG. 5B), as assessed by the concentrations of TNF in the supernatants determined by ELISA.

### Example 6

### Treatment of Inflammation as a Result of Trauma Using the Methods of the Invention

Trauma (e.g., head trauma, e.g., traumatic brain injury (TBI)) usually results from a violent impact, blow, or jolt. TBI, for example, usually results when a violent blow to the head causes the brain to collide with the inside of the skull (closed TBI) or when a foreign object (e.g., a bullet, knife) enters the skull and injures the brain. Humans of both sexes and all ages are susceptible to TBI, and non-fatal TBI may result in impaired cognitive function (e.g., attention, memory), motor function (e.g., extremity weakness, balance, coordination), sensation (e.g., hearing, vision, touch), and/or emotion (e.g., depression, anxiety, aggression).

Hemopexin may be administered to a subject with inflammation as a result of trauma (e.g., traumatic brain injury (TBI)). The Hx composition may be administered locally to a site of inflammation or systemically if necessary according to an appropriate administration route described herein. Upon administration to the subject with TBI, the Hx sequesters extravascular Hb present in non-vascular tissue as a result of broken blood vessels or necrosis following TBI and thereby reduces inflammation in the subject with TBI. Successful treatment may be measured by, e.g., a physician during a physical examination or by other tests and methods known in the art.

The dose of Hx or the number of treatments using Hx may be increased or decreased based on the severity of, occurrence of, or progression of, the inflammation or inflammation-related symptoms in the subject.

### Example 7

### Prophylaxis or Treatment of Inflammation in a Premature Infant Using the Methods of the Invention

Premature infants have a high incidence of inflammation and susceptibility for further inflammation (e.g., inflammation of the lungs, eyes, brain), often presenting with additional signs of toxicity and central nervous system bleeding. Inflammation of infectious and non-infectious etiology in premature infants can be a contributing factor to premature infant mortality or permanent disability. Approximately 1 in 10 non-fatal preterm births result in premature infants who develop permanent disabilities such as lung disease, cerebral palsy, blindness, or deafness.

Hemopexin may be administered to a premature infant to reduce or prevent inflammation. The Hx composition may be administered locally to a site of inflammation (e.g., administration directly to the eye to treat inflammation of the eye, such as retinopathy of prematurity (ROP)) or systemically (e.g., intravenous administration to treat inflammation resulting from bleeding) if necessary according to an appropriate administration route described herein. Upon administration to the premature infant, the Hx sequesters extravascular Hb present in non-vascular tissue, and thereby treats, reduces, or prevents one or more of the above-mentioned symptoms or permanent disabilities resulting from preterm birth. Successful treatment may be measured by, e.g., a physician during a physical examination or by other tests and methods known in the art.

The dose of Hx or the number of treatments using Hx may be increased or decreased based on the severity of, occurrence of, or progression of, the inflammation or inflammation-related symptoms in the premature infant.

### Example 8

### Treatment of Inflammation as a Result of Acute Lung Injury Using the Methods of the Invention

Acute lung injury (ALI) is a lung injury characterized by hypoxemia, non-cardiogenic pulmonary edema, and capillary leakage. ALI is a disorder of acute inflammation. The incidence of ALI in the U.S. is approximately 190,000 cases per year. ALI is principally caused by sepsis, but the stimulus of local or systemic inflammation need not be of infectious origin. The symptoms of ALI include breathing problems and rapid lung failure. The current treatment of ALI is based on ventilator and nonventilatory strategies. ALI has a mortality risk of approximately 29-42%.

Hemopexin may be administered to a subject with inflammation as a result of ALI of non-infectious origin. The Hx composition may be administered locally or systemically if necessary according to an appropriate administration route described herein. Upon administration to the subject with ALI, the Hx sequesters extravascular Hb present in non-vascular tissue, and thereby treats or reduces one or more of the above-mentioned symptoms of ALI in the subject. Successful treatment may be measured by, e.g., a physician during a physical examination or by other tests and methods known in the art.

The dose of Hx or the number of treatments using Hx may be increased or decreased based on the severity of, occurrence of, or progression of, the inflammation or inflammation-related symptoms in the subject.

### Example 9

### Treatment of Inflammation as a Result of Eye Conditions with Neovascularization Using the Methods of the Invention

Eye conditions with extravascular blood or inflammation (e.g., eye diseases with neovascularlization) include ocular trauma and numerous eye diseases such as age-related macular degeneration (AMD), myopia, diabetic retinopathy, and ROP. Ocular trauma and eye diseases associated with choroidal neovascularization can result in blurred vision, severely impaired sight, or complete blindness.

Hemopexin may be administered to a subject with inflammation as a result of ocular trauma or an eye disease associated with extravascular blood or inflammation. The Hx composition may be administered locally to a site of inflammation (e.g., administration to the eye) or systemically, if necessary, according to an appropriate administration route described herein. Upon administration to the subject with inflammation of the eye, the Hx sequesters extravascular Hb present in non-vascular tissue, and thereby treats or reduces one or more of the above-mentioned symptoms in the subject. Successful treatment may be measured by, e.g., a physician during a physical examination or by other tests and methods known in the art.

The dose of Hx or the number of treatments using Hx may be increased or decreased based on the severity of, occurrence of, or progression of, the inflammation or inflammation-related symptoms in the subject.

### Example 10

### Treatment of Inflammation as a Result of Acute Chest Syndrome Using the Methods of the Invention

Acute chest syndrome (ACS) is a common complication associated with subjects having sickle cell anemia. ACS accounts for approximately 25% of premature deaths of subjects with sickle cell anemia. Subjects with ACS commonly present with symptoms including fever, cough, chest pain, dyspnea, hypoxia, leukocytosis, and infiltrates on chest radiographs. Although ACS is often associated with an underlying infection, some cases of ACS present with no related infection or no diagnosis of a related infection. The present invention encompasses treatment of a subject with ACS with no apparent relation to infection.

Hemopexin may be administered to a subject with inflammation as a result of ACS. The Hx composition may be administered locally to a site of inflammation or systemically if necessary according to an appropriate administration route described herein. Upon administration to the subject with ACS, the Hx sequesters extravascular Hb present in non-vascular tissue, and thereby treats or reduces one or more of the above-mentioned symptoms of ACS in the subject. Successful treatment may be measured by, e.g., a physician during a physical examination or by other tests and methods known in the art.

The dose of Hx or the number of treatments using Hx may be increased or decreased based on the severity of, occurrence of, or progression of, the inflammation or inflammation-related symptoms in the subject.

### Example 11

### Treatment of Inflammation as a Result of Sickle Cell Anemia Using the Methods of the Invention

Hemopexin may be administered to a subject with inflammation as a result of sickle cell anemia. The Hx composition may be administered locally to a site of inflammation or systemically if necessary according to an appropriate administration route described herein. Upon administration to the subject with sickle cell anemia, the Hx sequesters extracellular Hb, and thereby treats or reduces, in the subject, an inflammation resulting from sickle cell anemia. Successful treatment may be measured by, e.g., a physician during a physical examination or by other tests and methods known in the art.

The dose of Hx or the number of treatments using Hx may be increased or decreased based on the severity of, occurrence of, or progression of, the inflammation or inflammation-related symptoms in the subject.

### Example 12

### Treatment of Inflammation as a Result of Asthma Using the Methods of the Invention

Asthma is a common chronic inflammatory disease of the airways. Asthma is characterized by reversible airflow obstruction, bronchospasm, and variable and recurring symptoms. The symptoms of asthma include wheezing, coughing, shortness of breath, fatigue, and chest tightness. As of 2009, more than 300 million people worldwide (5-10% of the world population) are affected by asthma. Over 250,000 deaths per year are attributed to asthma. The current treatment of acute asthma symptoms is usually with an inhaled short-acting beta-2 agonist (e.g., salbutamol/Albuterol).

Asthma is mediated by leukotriene B4 (LTB4), a fatty signaling molecule, which contributes to the pathophysiology of asthma, causing or potentiating bronchoconstriction, mucosal accumulation, airflow obstruction, increased secretion of mucus, and infiltration of inflammatory cells in the airway wall. Recent data suggest that the activation of LTB4 synthesis by macrophages is induced by heme (Monteiro et al., J. Immunol, 2011, 186: 6562-6567). Therefore, Hx may be administered to treat a subject with asthma.

The Hx composition may be administered locally to a site of inflammation or systemically if necessary according to an appropriate administration route described above. Upon administration to the subject with asthma, the Hx sequesters extravascular Hb present in non-vascular tissue, and thereby treats or reduces one or more of the above-mentioned symptoms of asthma in the subject. Successful treatment may be measured by, e.g., a physician during a physical examination or by other tests and methods known in the art.

The dose of Hx or the number of treatments using Hx may be increased or decreased based on the severity of, occurrence of, or progression of, the inflammation or inflammation-related symptoms in the subject.

### Example 13

### Removing Extracellular Hb from Blood

Blood containing extracellular Hb can be contacted with a membrane containing human Hx to remove the extracellular Hb from the blood.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

U.S. provisional application No. 61/540,309, filed Sep. 28, 2011 and U.S. provisional application No. 61/651,349, filed May 24, 2012, are hereby incorporated by reference in their entirety. All publications and patent applications mentioned in this specification are herein incorporated by reference to the same extent as if each independent publication or patent application was specifically and individually indicated as being incorporated by reference in its entirety.

1. A method of sequestering extravascular hemoglobin, said method comprising administering human hemopexin, or an amino acid sequence that is at least 95% identical to human hemopexin, to a subject in an amount sufficient to sequester at least 20% of said extravascular hemoglobin, wherein said sequestering reduces inflammation in said subject or reduces the risk of said subject developing inflammation.

2. The method of claim 1, wherein said administering comprises administering human hemopexin.

3. The method of claim 1, wherein said administered human hemopexin sequesters at least 50% of said extravascular hemoglobin.

4. The method of claim 1, wherein said administered human hemopexin sequesters at least 80% of said extravascular hemoglobin.

5. The method of claim 1, wherein said administered human hemopexin sequesters at least 90% of said extravascular hemoglobin.

6. The method of claim 1, wherein said subject has suffered trauma.

7. The method of claim 6, wherein said trauma is an acute lung injury.

8. The method of claim 1, wherein said subject has suffered a stroke.

9. The method of claim 1, wherein said extravascular hemoglobin is present in an eye of said subject.

10. The method of claim 9, wherein said hemopexin is administered directly to the eye.

11. The method of claim 10, wherein said administration comprises direct injection into the eye.

12. The method of claim 1, wherein necrotic tissue is present in said subject.

13. The method of claim 12, wherein said administration comprises direct injection into said necrotic tissue.

14. The method of claim 1, wherein said subject is suffering from acute chest syndrome.

15. The method of claim 1, wherein said subject has asthma.

16. The method of claim 1, wherein said administration is parenteral administration.

17. The method of claim 1, wherein said administration is topical administration.

18. The method of claim 1, wherein said subject is a human.

19. The method of claim 18, wherein said human is a premature infant.

20. A method of removing extracellular or extravascular hemoglobin from blood or a tissue, said method comprising contacting said blood or tissue with a substrate comprising human hemopexin, wherein said contacting results in the removal of said extracellular or extravascular hemoglobin from said blood or tissue.

21. The method of claim 20, wherein said extracellular hemoglobin is removed from said blood prior to or during a blood transfusion.

22. The method of claim 20, wherein said extracellular or extravascular hemoglobin is removed from said blood or tissue to treat an inflammatory response in a subject.
